(19) United States
(12) Patent Application Publication
(10) Pub. No.: US 2008/0200807 A1
(43) Pub. Date: Aug. 21, 2008

(54) Attitude-sensing ultrasound probe

(75) Inventors: Jeffrey L. Wright, Plymouth Meeting, PA (US); Gregory C. Berlin, Spring City, PA (US)

Correspondence Address: MENDELSOHN AND ASSOCIATES, P.C., 1500 JOHN F. KENNEDY BLVD., SUITE 405, PHILADELPHIA, PA, 19102, US

(73) Assignee: Accutome Ultrasound, Inc., Malvern, PA (US)

(21) Appl. No.: 11/708,808

(22) Filed: Feb. 20, 2007

(51) Int. Cl. A61B 8/10

(52) U.S. Cl. 600/443

(57) ABSTRACT

In one embodiment, an apparatus for generating attitude data corresponding to B-scan imaging data generated by an ultrasound probe. The apparatus includes one or more magnetic field sensors generating, based on measurements of the earth's magnetic field, magnetic field data for the probe, which is used to determine the orientation of the probe in a plane. The apparatus preferably also includes one or more accelerometers for determining tilt orientation and position. The apparatus further includes an interface providing, based on the magnetic field data, output data corresponding to the imaging data. The apparatus can be integrated into a probe or can be an attachment to retrofit an existing probe.

### BACKGROUND OF THE INVENTION

1. Field of the Invention

The present invention relates to ultrasound imaging, and, in particular, to handheld probes for obtaining ultrasound images of a patient.

2. Description of the Related Art

Medical ultrasonography (sonography) is an ultrasound-based diagnostic imaging technique used to visualize muscles and internal organs, their size and structure, and any pathological lesions or other abnormalities. In medical ultrasonography, a sound wave is typically produced by creating short, strong pulses of sound from a phased array of piezoelectric transducers, which are ordinarily formed, e.g., from a type of ceramic. Alternatively, only a single transducer may be used in certain instances. The transducers and associated electrical wiring are encased in a probe. The electrical pulses vibrate the ceramic of the transducers to create a series of sound pulses from each transducer. The frequencies present in this sound wave are typically between 2 and 50 MHz, well above the capabilities of the human ear (hence, the term “ultrasound”). The goal is to produce a single focused arc-shaped sound wave from the sum of all the individual pulses emitted by the transducers.

To make sure the sound wave is transmitted efficiently into the body of a patient, the transducer face has a patient-contacting rubber or plastic coating or tip through which the ultrasound signals pass, and typically, a water-based gel is disposed between the coating or tip and the surface of the patient's body.

The sound wave is partially reflected from the interface between different tissues and returns to the transducer in the form of an echo. Sounds that are scattered by very small structures in the patient's body also produce echoes.

The return of the sound wave to the transducer results in a reverse process from that of the transmission of the sound wave. The return sound wave vibrates the transducer's elements, which convert the vibration into electrical pulses that are transmitted from the probe to an ultrasound scanner, where the pulses are processed and transformed into a digital image.

The ultrasound scanner uses software to determine from each received echo (i) which of the multiple transducer elements received the echo, (ii) the strength of the echo, and (iii) the length of time it took for the echo to be received relative to the time it was transmitted. With the foregoing data, the software in the ultrasound scanner determines which pixels in the resulting ultrasound image or images are to be illuminated/printed and the brightness/darkness of each such pixel.

To generate a two-dimensional image, the ultrasound beam is swept either electronically using the phased array of acoustic transducers contained in the probe, or mechanically by a human operator. During the sweep, a series of slices (cross-sectional views) are taken.

One type of two-dimensional ultrasonic imaging referred to as “B-scan ultrasonography” (or simply “B-scan”) is a diagnostic test used in ophthalmology to image the interior of the eye, typically producing a series of two-dimensional slices of the eye and the orbit.

In B-scan imaging, the operator orients a probe in a series of different positions to obtain images of the entire inside surface of the back of the eye. The operator manually records an indicator of the orientation of the probe for each image taken, usually by noting the location of a scan plane marker (usually a white dot or line) imprinted on the probe relative to the patient's eye.

Several categories of B-scan images can be obtained: axial (horizontal axial, vertical axial, and oblique axial), longitudinal, and transverse (horizontal transverse, vertical transverse, and oblique transverse). For each type of B-scan, the operator manually records the indicator of the orientation of the scan plane marker on the probe using the hours of an imaginary clock superimposed on an eye being examined, as shown in FIG. 1.

FIG. 2 shows the probe positioning for an axial probe scan, in which the slice obtained is aligned through the center of the lens of a patient's right eye. For this type of scan, the patient looks in primary gaze (straight ahead), and the probe face is centered on the cornea and tilted slightly toward the nose, so that the probe is aimed directly at the optic nerve. As shown in FIG. 2, axial scans may be taken horizontally, vertically, or obliquely. A horizontal axial scan (represented by plane “H” in FIG. 2) is accomplished by rotating the scan plane marker on the probe to aim toward the 3-o'clock position for the right eye or the 9-o'clock position for the left eye. This results in the slice cutting through the nerve horizontally. For a horizontal axial scan, the operator records an indicator of “3AX” for the right eye or “9AX” for the left eye, or “HAX” (“horizontal axial”) for either eye.

A vertical axial scan (represented by plane “V” in FIG. 2) is produced by rotating the scan plane marker superiorly toward the 12-o'clock position in either eye. The slice will now cut through the nerve vertically. For a vertical axial scan, the operator records “12AX” or “VAX” (“vertical axial”). For oblique axial scans (represented by exemplary planes “O” in FIG. 2), the scan plane marker on the probe is rotated to include the clock hour(s) desired. For an oblique axial scan, the operator records an indicator corresponding to the clock hour toward which the scan plane marker is oriented, e.g., “1:30AX” or “10:30AX.”

FIG. 3 shows the probe positioning for a longitudinal probe scan, in which each slice obtained is a radial scan. For this type of scan, the patient's gaze and the probe are directed toward the area of interest, while the probe contacts the opposite portion of the sclera (the white, protective, outer layer of the eyeball). The scan plane marker on the probe is directed toward the cornea, regardless of the clock hour being examined. For a longitudinal probe scan, the operator records an indicator of “10:30L” if the 10:30 hour is being examined, “3L” if the 3:00 hour is being examined, and so forth.

FIG. 4 shows the probe positioning for a transverse probe scan. For this type of scan, the patient's gaze and the probe are directed toward the area of interest, while the probe contacts the opposite portion of the sclera. The probe face is oriented so as to be parallel to the limbus (the junction of the cornea and the sclera). For a vertical transverse probe scan (represented by planes “V” in FIG. 4), the scan plane marker is aimed superiorly, i.e., at 12:00, so that the resulting view will show the superior portion of the globe.

For a horizontal transverse probe scan (represented by planes “H” in FIG. 4), the scan plane marker is aimed nasally (i.e., toward 3:00 in the right eye and 9:00 in the left eye), so that the resulting view will show the nasal section of the globe. For an oblique transverse probe scan (represented, e.g., by planes “O” in FIG. 4), the scan plane marker is aimed toward the upper portion of the globe, so that the resulting view will show the upper portion of the globe. For a transverse probe scan, the operator records an indicator consisting of the clock hour in the center on the right side, followed by an estimation of how far in the periphery the slice is at that clock hour. The labeling system for this estimation is as follows: “P” for posterior pole, “PE” for posterior/equator, “EP” for equator/posterior, “E” for equator, “EA” for anterior to the equator, “CB” for ciliary body (the circumferential tissue inside the eye composed of the ciliary muscle and ciliary processes), and “O” for ora serrata (the serrated junction between the retina and the ciliary body).

Using the foregoing-described B-scan methodology, once an image is acquired, there is no exact way to tell which part of the eye was imaged without using the operator's recordation of the probe's orientation. Thus, it can be seen that the reliability of the manual B-scan method is heavily operator-dependent. A high level of skill and experience is needed to acquire quality images that can be used to make accurate diagnoses. Even more importantly, a single image that is incorrectly labeled can easily lead to surgical errors or other treatment problems.

### SUMMARY OF THE INVENTION

Problems in the prior art are addressed in accordance with the principles of the present invention by providing an ultrasound probe that can detect its own attitude (i.e., position and/or orientation), thereby eliminating reliance on an operator's recordation of scan type and clock hour.

In one embodiment, the present invention provides an apparatus for generating data corresponding to attitude of a probe generating imaging data. The apparatus includes one or more magnetic field sensors and an interface. The one or more magnetic field sensors generate, based on measurements of the earth's magnetic field, magnetic field data for the probe. The interface provides, based on the magnetic field data, output data corresponding to the imaging data. The output data identifies the attitude of the probe during generation of the imaging data.

In another embodiment, the present invention provides a method for generating data corresponding to attitude of a probe generating imaging data. The method includes: generating, based on measurements of the earth's magnetic field, magnetic field data for the probe; and providing, based on the magnetic field data, output data corresponding to the imaging data, wherein the output data identifies the attitude of the probe during generation of the imaging data.

In a further embodiment, the present invention provides an apparatus for generating output data corresponding to attitude of a probe generating imaging data. The apparatus includes one or more sensors, a calibration switch, a processor, and an interface. The one or more sensors generate attitude measurements for the probe. The processor, upon activation of the calibration switch, identifies current attitude measurements as baseline attitude measurements. The interface provides the output attitude data based on subsequent attitude measurements and the baseline attitude measurements.

In yet a further embodiment, the present invention provides a method for generating output attitude data corresponding to imaging data generated by a probe. The method includes: generating attitude measurements for the probe; upon activation of a calibration switch, identifying current attitude measurements as baseline attitude measurements; and providing the output attitude data based on subsequent attitude measurements and the baseline attitude measurements.

### BRIEF DESCRIPTION OF THE DRAWINGS

Other aspects, features, and advantages of the present invention will become more fully apparent from the following detailed description, the appended claims, and the accompanying drawings in which like reference numerals identify similar or identical elements.

FIG. 1 illustrates a clock superimposed on a patient's eye, as used by an ultrasound operator to manually record the orientation of an ultrasound probe during a B-scan of the eye;

FIG. 2 illustrates graphically a plurality of possible probe positions during an axial probe B-scan of the eye;

FIG. 3 illustrates graphically a plurality of possible probe positions during a longitudinal probe B-scan of the eye;

FIG. 4 illustrates graphically a plurality of possible probe positions during a transverse probe B-scan of the eye;

FIG. 5 illustrates a side perspective view of an attitude-sensing ultrasound probe in one embodiment of the invention;

FIG. 6 illustrates a cutaway plan view of the internal components of the attitude-sensing ultrasound probe of FIG. 5;

FIG. 7 illustrates a cutaway end view of the internal components of the attitude-sensing ultrasound probe of FIG. 5;

FIG. 8 illustrates a block diagram of the system board of the attitude-sensing ultrasound probe of FIG. 5;

FIG. 9 illustrates a side perspective view of an attitude-sensing probe attachment clamped onto a conventional ultrasound probe in one embodiment of the invention;

FIG. 10 illustrates a cutaway end view of the internal components of the attitude-sensing probe attachment of FIG. 9; and

FIG. 11 illustrates a block diagram of the system board of the attitude-sensing probe attachment of FIG. 9.

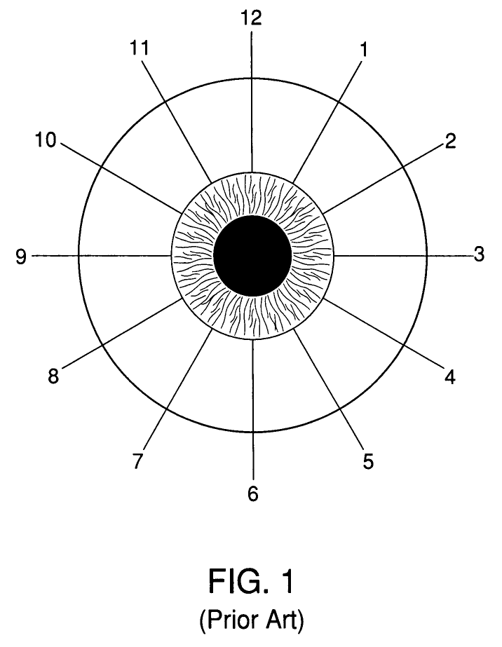

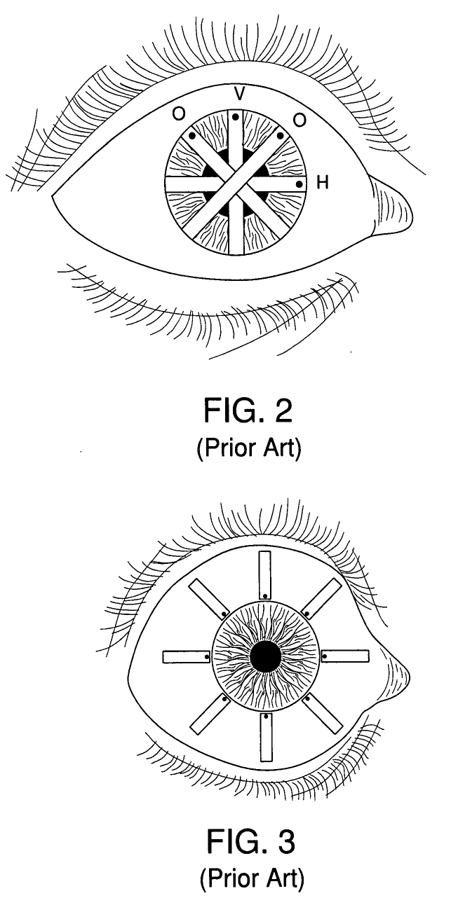

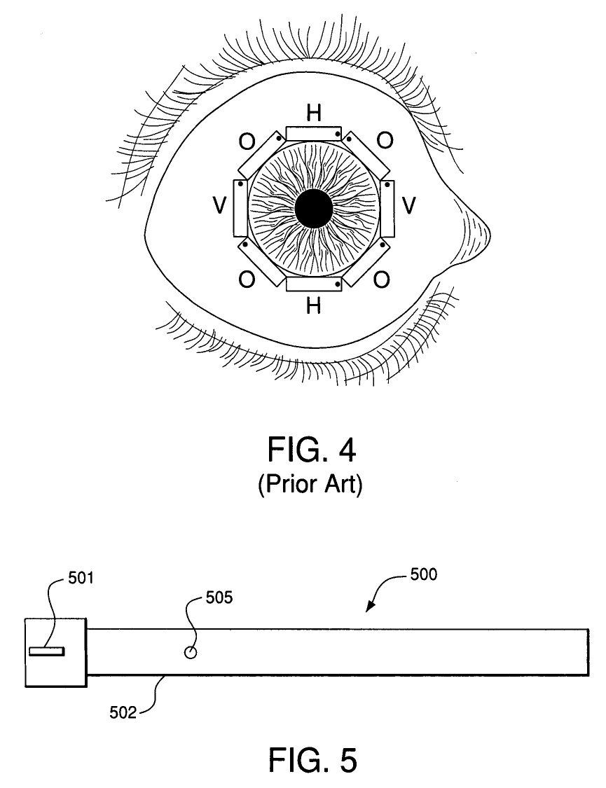

### DETAILED DESCRIPTION

FIG. 5 illustrates an attitude-sensing ultrasound probe 500 in one embodiment of the invention. As shown, the housing 502 of probe 500 is dimensioned and shaped like a conventional housing for an ultrasound probe that might be used to perform B-scan imaging and has a scan plane marker 501 located at one edge of the distal end of the probe. However, it should be understood that an ultrasound probe consistent with the present invention may have a housing of any suitable size, shape, and dimension.

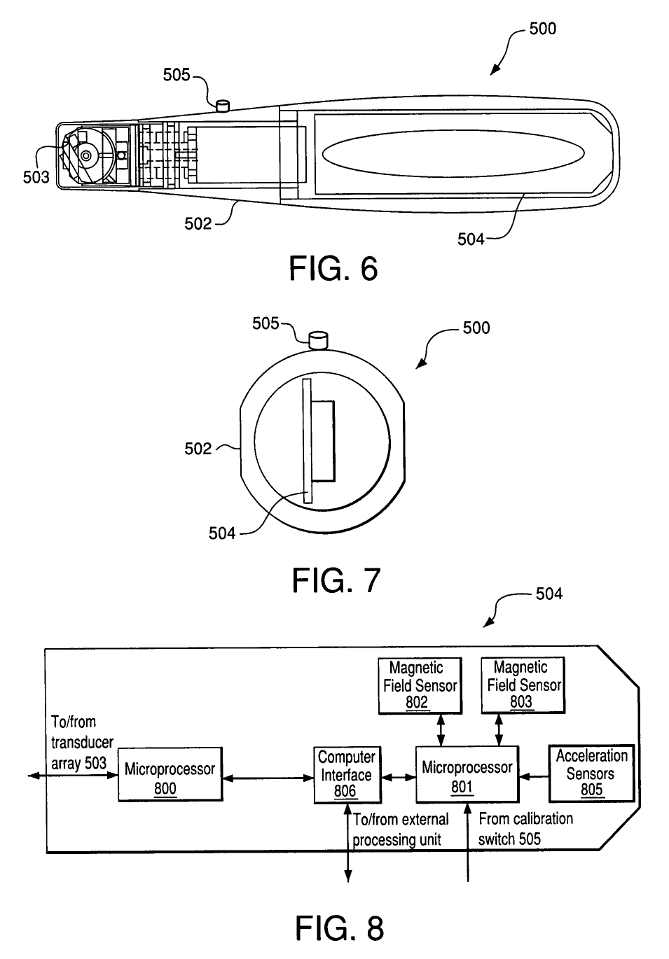

With reference now to FIG. 6 and FIG. 7, cutaway views showing the internal components of attitude-sensing ultrasound probe 500 are provided. The term “attitude-sensing,” as used herein, refers to the probe's ability to sense automatically its own position and/or orientation relative to a patient's eye, thereby reducing or eliminating the problems associated with the dependence in the prior art on an operator's recordation of a probe's scan type and clock hour. As shown in FIG. 6, probe 500 includes housing 502 containing a transducer array 503 and a system board 504. A momentary-contact calibration switch 505 is disposed on housing 502 and permits an operator to establish from the outset a baseline position and/or orientation of probe 500 with respect to a patient being imaged. Subsequent position and/or orientation measurements will be compared to these baseline measurements. Transducer array 503 is a standard array of transducer elements arranged in a predetermined configuration, with one or more signal cables (not shown) connected to each one of the ultrasound transducer elements to permit both transmission of electrical signals to respective transducer elements and receipt of electrical signals from respective transducer elements.

FIG. 8 illustrates the components of system board 504. As shown, system board 504 includes microprocessors 800 and 801, magnetic field sensors 802 and 803, acceleration sensors 805, and computer interface 806.

As with a conventional ultrasound probe, microprocessor 800 (i) transmits electrical signals to transducer array 503 to control the transmission of ultrasound signals by the transducer elements and (ii) receives electrical signals from transducer array 503 based on reflected ultrasound signals received by the transducer elements. To control the transmission of ultrasound signals by the transducer elements, microprocessor 800 uses control signals received from an external processing unit (not shown), such as an ultrasound scanner, via computer interface 806. Based on variances between the electrical signals transmitted to transducer array 503 and received from transducer array 503, microprocessor 800 generates imaging data signals, which microprocessor 800 provides to the external processing unit via computer interface 806.

Computer interface 806 couples attitude-sensing ultrasound probe 500 to the external processing unit, which (i) supplies control signals to microprocessor 800 to cause the transmission of ultrasound signals by transducer array 503 and (ii) receives data signals from microprocessor 800 based on electrical signals generated by transducer array 503. While computer interface 806 can be an interface to a custom or proprietary external processing unit, it should be understood that, in certain embodiments, computer interface 806 can be a conventional communications interface, such as a universal serial bus (USB) or Bluetooth interface, e.g., for interfacing with custom controller software executing on a conventional personal computer serving as an ultrasound scanner.

Each of magnetic field sensors 802 and 803 measures the force generated by the Earth's magnetic field as a magnetic source and outputs raw measurement data to microprocessor 801. Magnetic field sensors 802 and 803 are desirably passive magnetic tracking sensors or magnetometers in integrated-circuit form, situated so as to sense orientation in perpendicular directions from one another, relative to magnetic north. In combination, the data generated by magnetic field sensors 802 and 803 are used to determine the rotation of attitude-sensing ultrasound probe 500 in a plane.

Acceleration sensors 805 desirably include three accelerometers mounted orthogonally with respect to one another, so as to permit the measurement of pitch and roll tilt angles from the gravity vector. (Other non-orthogonal mounting configurations that span three-dimensional space are also possible.) Since gravity is a constant downward acceleration of approximately 9.8 meters/second² on Earth, the tilt orientation of attitude-sensing ultrasound probe 500 can be calculated based on measurements of the components of the gravity force that is being applied to the three accelerometers. These measurements are also used to track the translation of the probe, such that the probe can automatically detect (i) changes in position for different slices of a single eye and (ii) changes in position from imaging a patient's left eye to the patient's right eye, and vice-versa. In certain embodiments, the operator might be instructed to scan a patient's eyes in the same sequence every time, e.g., left eye first, then right eye. In other embodiments, the operator could simply use the calibration switch to register the change in obtaining images from one eye to the other, or could manually indicate in some other manner which eye is being scanned.

Microprocessor 801 is coupled to (i) receive a control signal from calibration switch 505 mounted on housing 502, indicating that current magnetic field and acceleration data should be stored as baseline measurements, and (ii) store these baseline measurements. Microprocessor 801 subsequently receives (i) raw magnetic field data from magnetic field sensors 802 and 803 and (ii) raw acceleration data from acceleration sensors 805, both of which microprocessor 801 compares to the stored baseline measurements to generate an indicator of the current orientation and position of attitude-sensing ultrasound probe 500.

In operation, prior to beginning the scanning process for a given patient, the operator depresses calibration switch 505 while holding probe 500 at a calibration position (e.g., at an oblique axial position, with the scan plane marker rotated to 12:00) with respect to the patient's eye being imaged. This baseline orientation and position of probe 500 at the time of calibration, as determined by magnetic field sensors 802 and 803 and acceleration sensors 805, are recorded, e.g., in a memory (not shown). Microprocessor 801 (i) compares all subsequent probe orientation and position measurements to the baseline orientation and position measurements stored during calibration and (ii) based on the comparison results, continuously generates the corresponding current orientation/position indicator. In one embodiment, each indicator is generated as a text string corresponding to the type of scan and clock hour, e.g., “9AX,” “10:30L,” and so forth. In alternative embodiments, the indicator might consist of magnetic field and acceleration data, either in raw form, or some other numeric representation, such that an external processing unit (instead of microprocessor 801) would process and convert the raw or numeric magnetic field and acceleration data into the appropriate text string. Thus, for each set of imaging data generated by microprocessor 800 and provided to the external processing unit via computer interface 806, a corresponding scan type and clock-hour indicator generated by microprocessor 801 is concurrently provided to the external processing unit. The indicator remains with its associated imaging data and can therefore be displayed and/or printed along with the image, resulting in the same type of indicator that would have otherwise been manually recorded by the operator, yet with greater accuracy and reliability due to the reduction or elimination of human error.

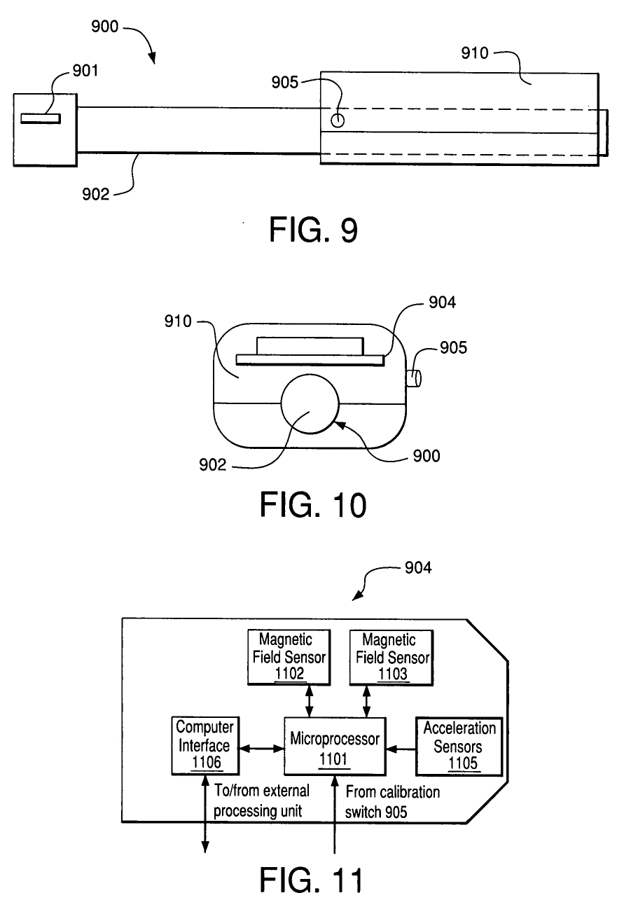

FIG. 9 illustrates an attitude-sensing probe attachment 910 in one embodiment of the invention, which is used in conjunction with and retrofits a conventional ultrasound probe 900. As shown, probe attachment 910 is clamped to a conventional ultrasound probe 900 that has a scan plane marker 901 located at one edge of the distal end of the probe. The housing of probe attachment 910 is dimensioned and shaped to fit around and clamp onto a portion of the cylindrical housing 902 of ultrasound probe 900. However, it should be understood that a probe attachment consistent with the present invention may be embodied in a housing of any suitable size, shape, and dimension and may attach to the housing of an ultrasound probe in ways other than the use of a clamping mechanism, e.g., using screws or other fasteners, adhesive, threading, compression fittings, etc.

With reference now to FIG. 10, a cutaway end view showing the internal components of attitude-sensing probe attachment 910 is provided. As shown, the components of probe attachment 910 are substantially the same as those of probe 500 (of FIG. 5, FIG. 6, FIG. 7, and FIG. 8), except that probe attachment 910 does not include a transducer array (503) or a second microprocessor (800), since probe 900 already includes these components. As with probe 500, probe attachment 910 includes (i) a momentary-contact calibration switch 905 that permits an operator to establish a baseline position and orientation of probe 500 with respect to a patient being imaged and (ii) a system board 904.

FIG. 11 illustrates the components of system board 904. As shown, system board 904 includes microprocessor 902, magnetic field sensors 1102 and 1103, acceleration sensors 1105, and computer interface 1106, all of which function in like manner to the corresponding components of probe 500. The principal difference between probe attachment 910 and probe 500 is that probe attachment 910 does not perform any processing relating to patient imaging data and processes only magnetic field and acceleration data, which probe attachment 910 provides via computer interface 1106 to a custom or proprietary external processing unit (not shown), such as an ultrasound scanner. The external processing unit also (i) receives patient imaging data from probe 900 and (ii) coordinates the storage of received magnetic field and acceleration data with the corresponding patient imaging data. This can be done by time-stamping the magnetic field and acceleration data as it is received by the external processing unit, so that the corresponding current orientation/position indicator can be appended to time-stamped patient imaging data, either as a text string corresponding to the type of scan and clock hour, e.g., “9AX,” “10:30L,” or as raw or numeric magnetic field and acceleration data. Thus, for each set of imaging data generated by probe 900, a corresponding scan type and clock-hour indicator generated by microprocessor 1101 is concurrently provided to the external processing unit. The indicator remains with its associated imaging data and can therefore be displayed and/or printed along with the image, resulting in the same type of indicator that would have otherwise been manually recorded by the operator, yet with greater accuracy and reliability due to the reduction of human error. Thus, the use of probe attachment 910 permits a conventional ultrasound probe (as well as other related ultrasound equipment) to be used without requiring the operator to record manually the type of scan and clock hour.

In certain embodiments, instead of or in addition to using magnetic field and/or acceleration data to create indicators for attachment to corresponding patient imaging data, the magnetic field and/or acceleration data could serve other purposes. For example, the magnetic field and/or acceleration data could be provided, along with the corresponding patient imaging data, to a processor adapted to generate one or more three-dimensional representations of various portions of the patient's eye from this data.

It should be understood that various functions of an attitude-sensing ultrasound probe or probe attachment may be carried out by a variety of different types of sensors, including one or more of mechanical trackers, accelerometers, gyroscopes, ultrasonic trackers, passive magnetic trackers, magnetometers, active magnetic trackers, global-positioning sensor (GPS) trackers, optical trackers, or similar devices.

It should be recognized that apparatus consistent with certain embodiments of the present invention may be used in the context of ultrasound imaging for non-eye organs and bodily regions, as well as in the context of imaging methods other than ultrasound.

It should further be recognized that, although certain embodiments of a probe consistent with the invention are described herein as having two microprocessors (e.g., microprocessors 800 and 801), a single microprocessor could alternatively provide the same functionality.

While calibration of a probe or probe attachment is described herein as employing a calibration switch, it should be understood that a mouse click or other switching means could alternatively be used to perform the same function.

Portions of the present invention may be implemented as circuit-based processes, including possible implementation as a single integrated circuit (such as an ASIC or an FPGA), a multi-chip module, a single card, or a multi-card circuit pack. As would be apparent to one skilled in the art, various functions of circuit elements may also be implemented as processing blocks in a software program. Such software may be employed in, for example, a digital signal processor, micro-controller, or general-purpose computer.

The present invention can be embodied in the form of methods and apparatuses for practicing those methods. The present invention can also be embodied in the form of program code embodied in tangible media, such as magnetic recording media, optical recording media, solid state memory, floppy diskettes, CD-ROMs, hard drives, or any other machine-readable storage medium, wherein, when the program code is loaded into and executed by a machine, such as a computer, the machine becomes an apparatus for practicing the invention. The present invention can also be embodied in the form of program code, for example, whether stored in a storage medium, loaded into and/or executed by a machine, or transmitted over some transmission medium or carrier, such as over electrical wiring or cabling, through fiber optics, or via electromagnetic radiation, wherein, when the program code is loaded into and executed by a machine, such as a computer, the machine becomes an apparatus for practicing the invention. When implemented on a general-purpose processor, the program code segments combine with the processor to provide a unique device that operates analogously to specific logic circuits.

The present invention can also be embodied in the form of a bitstream or other sequence of signal values electrically or optically transmitted through a medium, stored magnetic-field variations in a magnetic recording medium, etc., generated using a method and/or an apparatus of the present invention.

It will be further understood that various changes in the details, materials, and arrangements of the parts which have been described and illustrated in order to explain the nature of this invention may be made by those skilled in the art without departing from the scope of the invention as expressed in the following claims.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present invention.

Although the elements in the following method claims are recited in a particular sequence with corresponding labeling, unless the claim recitations otherwise imply a particular sequence for implementing some or all of those elements, those elements are not necessarily intended to be limited to being implemented in that particular sequence.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

We claim:

1. Apparatus for generating data corresponding to attitude of a probe generating imaging data, the apparatus comprising:
one or more magnetic field sensors adapted to generate, based on measurements of the earth's magnetic field, magnetic field data for the probe; and
an interface adapted to provide, based on the magnetic field data, output data corresponding to the imaging data, wherein the output data identifies the attitude of the probe during generation of the imaging data.

2. The invention of claim 1, further comprising one or more acceleration sensors adapted to generate acceleration data corresponding to the imaging data and further identifying the attitude of the probe, wherein the output data provided by the interface is further based on the acceleration data.

3. The invention of claim 1, wherein:
the apparatus comprises the probe; and
the probe, the one or more magnetic field sensors, and the interface are integrated in a single device.

4. The invention of claim 1, wherein:
the apparatus is adapted to be removably attached to the probe; and
the one or more magnetic field sensors and the interface are integrated in a single device that is adapted to retrofit an existing probe.

5. The invention of claim 1, further comprising a calibration switch, wherein the apparatus is adapted, upon activation of the calibration switch, to identify current magnetic field data as baseline magnetic field data.

6. The invention of claim 1, further comprising a processor adapted to use the magnetic field data to generate an indicator of at least one of position and orientation relative to an eye being imaged by the probe.

7. The invention of claim 1, further comprising:
a calibration switch; and
a processor adapted:
(i) upon activation of the calibration switch, to identify current magnetic field data as baseline magnetic field data, and
(ii) to generate subsequent output data by comparing subsequent measurements of the earth's magnetic field to the baseline magnetic field data.

8. The invention of claim 1, wherein:
the one or more magnetic field sensors comprise two magnetic field sensors disposed so as to sense orientation in two substantially mutually-perpendicular directions relative to the earth's magnetic north; and
the magnetic field data indicates orientation of the probe in a plane.

9. The invention of claim 1, wherein the interface is (i) a universal serial bus (USB) interface for interfacing with a computer having a USB bus or (ii) a wireless interface.

10. The invention of claim 1, wherein the output data comprises one or more indicators corresponding to scan type and clock hour for a B-scan of an eye.

11. The invention of claim 1, further comprising:
one or more acceleration sensors adapted to generate acceleration data corresponding to the imaging data and further identifying the attitude of the probe, wherein the output data provided by the interface is further based on the acceleration data;
a calibration switch; and
a processor for processing the magnetic field data and the acceleration data, wherein the processor is adapted:
(i) upon activation of the calibration switch, to identify (1) current magnetic field data as baseline magnetic field data and (2) current acceleration data as baseline acceleration data, and
(ii) to generate subsequent output data by comparing (1) subsequent magnetic field data to the baseline magnetic field data and (2) subsequent acceleration data to the baseline acceleration data.

12. The invention of claim 11, wherein the processor is adapted to generate and output an indicator of position and orientation relative to an eye being imaged by the probe based on the comparisons of (1) the subsequent magnetic field data with the baseline magnetic field data and (2) the subsequent acceleration data with the baseline acceleration data.

13. A method for generating data corresponding to attitude of a probe generating imaging data, the method comprising:
generating, based on measurements of the earth's magnetic field, magnetic field data for the probe; and
providing, based on the magnetic field data, output data corresponding to the imaging data, wherein the output data identifies the attitude of the probe during generation of the imaging data.

14. The invention of claim 13, further comprising generating acceleration data corresponding to the imaging data and further identifying the attitude of the probe, wherein the output data is further based on the acceleration data.

15. The invention of claim 13, further comprising identifying current magnetic field data as baseline magnetic field data based on activation of a calibration switch.

16. The invention of claim 13, further comprising processing the magnetic field data to generate an indicator of orientation relative to one or more body parts being imaged by the probe.

17. The invention of claim 16, wherein the one or more body parts is an eye.

18. The invention of claim 13, further comprising:
(i) identifying current magnetic field data as baseline magnetic field data based on activation of a calibration switch; and
(ii) generating subsequent output data by comparing subsequent measurements of the earth's magnetic field to the baseline magnetic field data.

19. The invention of claim 13, wherein:
(i) the step of generating the magnetic field data for the probe comprises sensing orientation in two substantially mutually-perpendicular directions relative to the earth's magnetic north; and
(ii) the magnetic field data indicates orientation of the probe in a plane.

20. The invention of claim 13, further comprising providing the output data (i) via a universal serial bus (USB) interface to a computer having a USB bus or (ii) via a wireless interface.

21. The invention of claim 13, wherein the output data comprises one or more indicators corresponding to scan type and clock hour for a B-scan of an eye.

22. The invention of claim 13, wherein the output data is generated without inducing a magnetic field that substantially affects the magnetic field data, such that the only substantial magnetic field used to generate the output data is the earth's magnetic field.

23. The invention of claim 13, further comprising:
generating acceleration data (i) corresponding to the imaging data and (ii) further identifying the attitude of the probe, wherein the output data is further based on the acceleration data;
upon activation of a calibration switch, identifying (1) current magnetic field data as baseline magnetic field data and (2) current acceleration data as baseline acceleration data, and
generating subsequent output data by comparing (1) subsequent magnetic field data to the baseline magnetic field data and (2) subsequent acceleration data to the baseline acceleration data.

24. The invention of claim 23, further comprising generating and outputting an indicator of position and orientation relative to an eye being imaged by the probe based on the comparisons of (1) the subsequent magnetic field data with the baseline magnetic field data and (2) the subsequent acceleration data with the baseline acceleration data.

25. Apparatus for generating output data corresponding to attitude of a probe generating imaging data, the apparatus comprising:
one or more sensors adapted to generate attitude measurements for the probe;
a calibration switch;
a processor adapted, upon activation of the calibration switch, to identify current attitude measurements as baseline attitude measurements; and
an interface adapted to provide the output attitude data based on subsequent attitude measurements and the baseline attitude measurements.

26. The invention of claim 25, wherein:
the processor compares the subsequent attitude measurements to the baseline attitude measurements to determine position and orientation of the probe; and
the output attitude data comprises one or more indicators corresponding to scan type and clock hour for a B-scan of an eye.

27. A method for generating output attitude data corresponding to imaging data generated by a probe, the method comprising:
generating attitude measurements for the probe;
upon activation of a calibration switch, identifying current attitude measurements as baseline attitude measurements; and
providing the output attitude data based on subsequent attitude measurements and the baseline attitude measurements.

28. The invention of claim 27, further comprising:
comparing the subsequent attitude measurements to the baseline attitude measurements to determine position and orientation of the probe, wherein the output attitude data comprises one or more indicators corresponding to scan type and clock hour for a B-scan of an eye.
